(19) United States
(12) Patent Application Publication
(10) Pub. No.: US 2007/0135910 A1
(43) Pub. Date: Jun. 14, 2007

(54) Vascular prosthesis

(76) Inventor: Eugene Bell, Boston, MA (US)

Correspondence Address: FISH & RICHARDSON PC, P.O. BOX 1022, MINNEAPOLIS, MN, 55440-1022, US

(21) Appl. No.: 11/484,903

(22) Filed: Jul. 12, 2006

Related U.S. Application Data
(60) Provisional application No. 60/698,606, filed on Jul. 12, 2005.

(51) Int. Cl. A61F 2/06

(52) U.S. Cl. 623/1.47; 264/281; 623/901

(57) ABSTRACT

A vascular prosthesis having one or more inner layers formed of an air-dried collagen-containing matrix and one or more outer layers formed of a freeze-dried collagen-containing matrix is described.

### CROSS-REFERENCE TO RELATED APPLICATIONS

This application claims the benefit of priority of U.S. application Ser. No. 60/698,606, filed on Jul. 12, 2005, the contents of which are hereby incorporated by reference in their entirety.

### TECHNICAL FIELD

This invention relates to tissue prostheses, and more particularly to vascular prostheses.

### BACKGROUND

Vascular prostheses have many applications, including vein and artery grafts. Vascular prostheses have been constructed from various non-biological materials, e.g., Dacron™, and from various biological materials. In addition, processed and non-processed vessels and arteries grafted from human and non-human sources are sometimes used as vascular prostheses.

It is desirable to have a vascular prosthesis that is biological compatible and does not leak or become stenotic or occluded.

### SUMMARY

Described herein is a vascular prosthesis having one or more inner layers formed of an air-dried collagen-containing matrix and one or more outer layers formed of a freeze-dried collagen-containing matrix. One suitable collagen-containing matrix is human or non-human, fetal or neonatal skin that has been processed to substantially remove the epidermal and subcutaneous layers. The vascular prosthesis can be constructed by wrapping a strip or a sheet of air-dried and rehydrated collagen-containing matrix one or more times around a suitable mandrel to create an inner layer or layers and then wrapping a strip or a sheet of freeze-dried and rehydrated collagen-containing matrix one or more times around the inner layer or layers of air-dried and rehydrated collagen-containing matrix to create an outer layer or layers. It has been found that this combination of inner and outer layers provides a prosthesis that is relatively impermeable to blood flowing within the prosthesis while allowing tissue ingrowth into the outer layer or layers of the prosthesis. The inner surface (luminal wall) of the prosthesis is expected to support growth and attachment of epithelial cells (e.g., epithelial cells arising from epithelial cells or immature cells seeded on the luminal wall), while being relatively impermeable to blood. In addition, it appears that the process of air-drying greatly reduces the ability of blood cells to bond to the collagen matrix forming the luminal wall of the prosthesis. The outer layer (or layers) is expected to permit vascularization and tissue cell penetration once the prosthesis has been implanted. The completed prosthesis can be air-dried and removed from the mandrel and sterilized for storage. The air-drying of the completed prosthesis promotes adherence of the various layers of collagen-containing matrix to adjacent layers of collagen-containing matrix, particularly when the layers are urged against each other.

The dried prosthesis is rehydrated before implanting into a patient.

It has also been found that using an expandable mandrel, e.g., a tube formed from an elastomeric material such as silicone facilitates production of the prosthesis. Such a mandrel can be adjusted to have a smaller diameter after the prosthesis has been produced in order to remove the mandrel from the prosthesis readily without causing damage to the prosthesis. As described in greater detail below, the elastomeric tube serving as a mandrel can be pressurized to expand its diameter before the various layers of collagen containing matrix are wrapped around it. After the prosthesis is completed and air dried, the pressure in the mandrel is released, reducing its diameter. The prosthesis can now be readily removed from the mandrel. Alternatively the tubular mandrel can be pressurized after some or all of the layers of collagen matrix have been applied. When the mandrel is a solid tube is pressurized by compressing the tube perpendicular to its length (e.g., by pressing inward along the length of the tube from both ends. Alternatively, a hollow tube can be inflated with air or a fluid to increase its diameter.

Described herein is a vascular prosthesis in the form of a tube comprising at least one layer of freeze-dried collagen-containing matrix surrounding at least one layer of air-dried collagen-containing matrix. In various embodiments: the vascular prosthesis comprises at least one layer of freeze-dried collagen-containing matrix surrounding at least two layers of air-dried collagen-containing matrix; the vascular prosthesis comprises at least two layers of freeze-dried collagen-containing matrix surrounding at least two layers of air-dried collagen-containing matrix; at least a portion of a first layer of air-dried collagen-containing matrix is mechanically joined to at least a portion of a second layer of air-dried collagen-containing matrix; at least a portion of a first layer of freeze-dried collagen-containing matrix is mechanically joined to at least a portion of a second layer of freeze-dried collagen-containing matrix; the first and second layers are mechanically joined by stitching; by dehydration and under pressure, the first and second layers are mechanically joined by stapling. The vascular prosthesis can have any desirable inner diameter, e.g., an inner diameter of: no more than 4 mm, 4-5 mm, 5-610 mm, at least 5 mm, at least 6 mm, at least 7 mm, no more than 8 mm, or at least 8 mm.

In various embodiments: the air-dried collagen-containing matrix can comprise non-human, fetal or neo-natal tissue; the freeze-dried collagen-containing matrix can comprise non-human, fetal or neo-natal tissue; and the air-dried collagen-containing matrix and the freeze-dried collagen-containing matrix comprises non-human, fetal or neo-natal tissue is bovine dermis that is substantially free of epidermal matter and subdermal matter.

In some cases a prosthesis can include one or more layers of air-dried collagen containing matrix and no layers of freeze-dried collagen containing matrix. Such a prosthesis can be formed by wrapping a strip or sheet of rehydrated air-dried collagen-containing matrix around a mandrel to create at least one layer of rehydrated air-dried collagen-containing matrix and then drying the prosthesis so formed. The mandrel can have a greater diameter before the rehydrated air-dried collagen-containing matrix is wrapped around a mandrel and a smaller diameter after the of air-dried collagen-containing matrix has been dried.

Also described is a method for forming a vascular prosthesis comprising:

(a) wrapping a strip or sheet of rehydrated air-dried collagen-containing matrix around a mandrel to create at least one layer of rehydrated air-dried collagen-containing matrix;

(b) wrapping a strip or sheet of rehydrated freeze-dried collagen-containing matrix around the at least one layer of rehydrated air-dried collagen-containing matrix to create at least one layer of rehydrated freeze-dried collagen-containing matrix surrounding the at least one layer of rehydrated air-dried collagen-containing matrix to create a vascular prosthesis; and

(c) removing the vascular prosthesis from the mandrel.

In certain cases, step (a) comprises wrapping the strip or sheet of rehydrated air-dried collagen-containing matrix around a mandrel to create at least two layers of rehydrated air-dried collagen-containing matrix. In certain cases, step (b) comprises wrapping the strip or sheet of rehydrated freeze-dried collagen-containing matrix around the at least one layer of rehydrated air-dried collagen-containing matrix to create at least two layers of rehydrated freeze-dried collagen-containing matrix surrounding the at least one layer of rehydrated air-dried collagen-containing matrix.

In some cases the collagen-containing matrix is in the form of a sheet large enough to wrap around a desired length of mandrel without the need for spiral wrapping. In other cases, for example when the strip or sheet of collagen matrix is not large enough to wrap around the mandrel without the need for spiral wrapping, the sheet or strip of collagen matrix material is spirally wrapped around the mandrel such that one gyre slightly overlaps the previous gyre thereby covering the desired length of mandrel. This spiral wrapping can repeated so that there are two spirally wrapped layers of material. Where spiral wrapping is used the number of layers of material will vary along the length of the mandrel.

In some cases the two or more of the at least two layers of rehydrated air-dried collagen-containing matrix are mechanically joined together and/or two or more of the at least two layers of rehydrated freeze-dried collagen-containing matrix are mechanically joined together. Layers can be mechanically joined by stitching, or by stapling or by any other convenient means. As noted above, air-drying of the completed prosthesis encourages adherence of the layers of collagen containing matrix to adjacent layers of collagen-containing matrix. In addition, fibrin glues can be used to increase adherence between layers.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A and 1B are schematic drawings of one way in which hydrated air-dried collagen containing matrix can be wrapped around a mandrel.

FIGS. 2A and 2B are schematic drawings of an alternative way in which hydrated air-dried collagen containing matrix can be wrapped around a mandrel.

Like reference symbols in the various drawings indicate like elements.

### DETAILED DESCRIPTION

Any suitable collagen-containing matrix can be used to create the air-dried collagen-containing matrix and freeze-dried collagen-containing matrix used to form the vascular prosthesis. One suitable collagen-containing matrix is fetal or neo-natal, human or non-human (e.g., porcine or bovine) dermis that is substantially free of epidermal matter and subdermal matter. The preparation of one such suitable material, called EBM, is described in U.S. Pat. No. 6,696,074.

### EXAMPLE 1

Various collagen-containing matrixes can be used to prepare a prosthesis. EBM is one suitable collagen-containing matrix. Air-dried and freeze-dried EBM can be prepared in various ways. For example, to generate EBM, a piece of fetal (e.g., from a 22-23 week fetus) bovine skin substantially free of pigment or holes is rinsed in water and then processed to substantially remove the epidermal and subcutaneous layers. The resulting material which is now primarily dermal tissue is soaked in 20% bleach for approximately 30 minutes on ice with gentle rocking. Generally about 0.1 ml of bleach is used per cm² of dermal tissue. The dermal tissue is then washed in water for about 20 minutes on ice.

After washing, the dermal tissue is placed on a flat, plastic plate epidermal side down. Any remaining subdermal flesh is removed by drawing a flat blade over the surface of the tissue. The pressure applied in this step can expel components released from the collagen matrix by previous processing steps, including undesirable material. The dermal tissue is turned over and remaining epidermal tissue is removed with a flat blade. The subdermal and epidermal tissue can also be removed using a slicing machine or a defleshing machine.

Next, the dermal tissue is placed in a 5.2N solution of NaOH on ice and rocked gently for about 15 minutes. In this step it can be desirable to provide approximately 1.0 ml of solvent per cm² of dermal tissue.

The NaOH treatment can be repeated one or more times with each treatment followed by applying pressure to the tissue, for example, as described above to remove components released from the tissue by the previous processing steps. The concentration of NaOH and the length of the exposure to the NaOH can be increased or decreased depending upon the thickness of the tissue. The tissue is then washed at least twice in water with each wash lasting at least about 15 minutes. This washing can optionally be followed by applying pressure to the tissue, for example, as described above to remove components released from the tissue by previous processing steps.

Next, the dermal tissue is treated with an organic solvent to substantially remove lipids. The tissue can washed in a 1:1 chloroform:ethanol for approximately 5-10 minutes and then in 70% ethanol for approximately 10-20 minutes. This is followed by two approximately 10 minute washes in water. This washing can optionally be followed by applying pressure to the tissue, for example, as described above to remove separated components from the tissue.

The dermal tissue prepared in this manner can be air-dried, for example, between two porous plates with or without the application of pressure until no longer tacky to create air-dried collagen-containing matrix.

To create freeze-dried collagen-containing matrix the dermal tissue prepared as described above is freeze-dried using standard conditions.

To form a vascular prosthesis, air-dried collagen-containing matrix prepared as described above, referred to as air-dried EBM, is first rehydrated by soaking in water or a saline solution, e.g., PBS to create rehydrated, air-dried EBM. A strip or sheet of this material is wrapped around a hollow expandable tube that having, for example, an approximately 6 mm outer diameter (when not unexpanded) to create at least one layer of rehydrated, air-dried EBM. Before this wrapping step takes place, the mandrel is pressurized to expand its diameter beyond its non-pressurized diameter. As explained in greater detail below, this step allows the completed and dried prosthesis to be later readily removed from the mandrel by releasing the pressure on the silicone tube, thus reducing the diameter of the mandrel. After the rehydrated, air-dried EBM is wrapped around the mandrel, a strip or sheet of freeze-dried collagen-containing matrix prepared as described above and subsequently rehydrated, referred to as rehydrated, freeze-dried EBM, is wrapped around the layers of rehydrated, air-dried EBM to create at least one layer of rehydrated, freeze-dried EBM. The prosthesis is allowed to air dry thoroughly until no longer tacky and is then removed from the mandrel by first releasing the pressure from the mandrel and then sliding the prosthesis from the mandrel. The prosthesis can be stored dried and then rehydrated in a suitable buffer before use.

In some cases a sheet of collagen-containing matrix will be large enough to wrap around the mandrel once creating a complete layer. If a sheet is smaller, it can be spirally wrapped around the mandrel with each wrapping slightly overlapping the previous wrapping. In this case the number of layers of air-dried or freeze dried collagen-containing matrix or both may vary along the length of the prosthesis. It can be desirable to create a prosthesis in which the sheet of hydrated air-dried collagen-containing matrix used to create that inner layer or layers is large enough to create a complete layer each time it is wrapped around the mandrel.

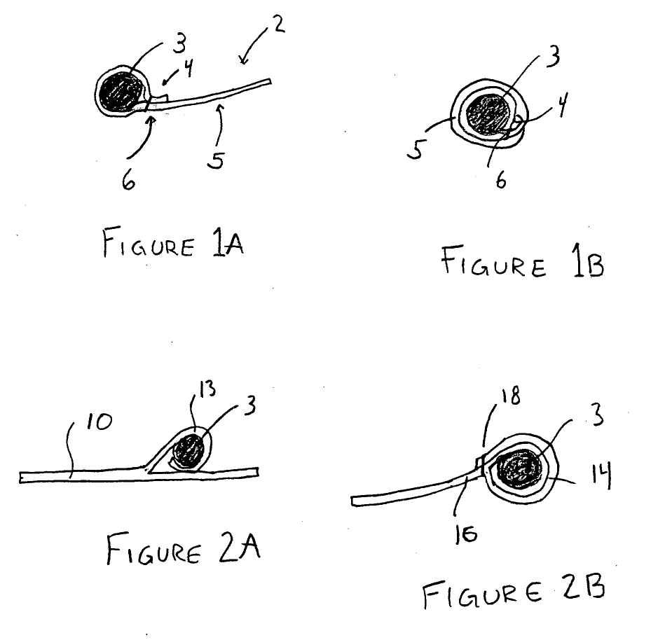

FIGS. 1A and 1B depict one method for wrapping a sheet of hydrated air-dried collagen-containing matrix around a mandrel. Referring to FIG. 1A, the sheet of hydrated air-dried collagen-containing matrix 2 is wrapped once around a pressurized mandrel 3 to create a first layer of hydrated air-dried collagen-containing matrix and leaving a small flap 4 of the sheet of hydrated air-dried collagen-containing matrix extending beyond the mandrel at one edge of the sheet hydrated air-dried collagen-containing matrix and a larger flap 5 of the sheet extending beyond the mandrel at the other edge of the sheet. The sheet of hydrated air-dried collagen-containing matrix is sewn to itself along the length of the mandrel using a suitable suturing material 6. The larger flap 5 is free to be wrapped around the mandrel to create a second complete layer of hydrated air-dried collagen-containing matrix as shown in FIG. 1B. Referring now to FIG. 1B, the larger flap 5 is wrapped around the mandrel 3 to create a second complete layer of hydrated air-dried collagen-containing matrix surrounding the first layer of hydrated air-dried collagen-containing matrix.

FIGS. 2A and 2B depict another method for wrapping a sheet of hydrated air-dried collagen-containing matrix around a mandrel using a sheet of hydrated air-dried collagen-containing matrix that has been split at one edge to create two half thickness of hydrated air-dried collagen-containing matrix. Referring to FIG. 2A, a sheet of hydrated air-dried collagen-containing matrix 10 has been split at one edge to create two half thickness of hydrated air-dried collagen-containing matrix, an upper half thickness 12 and a lower half thickness 14. The upper half thickness 12 is wrapped around the mandrel to create one layer of hydrated air-dried collagen-containing matrix 15. Referring to FIG. 2B the lower half thickness 14 is then wrapped around the mandrel and is stitched to the remaining full thickness portion of the hydrated air-dried collagen-containing matrix 16 along the entire length of the mandrel using a suitable suturing material 18.

A vascular prosthesis prepared as described above was tested using an in vitro vascular loop containing whole human blood for up to three weeks. No stenosis or clotting was observed. The prosthesis remained impermeable and the inner surface of the prosthesis was not damaged as determined histologically. When blood leakage was measured, this composite vascular prosthesis did not leak fluid or circulating blood cells. A vascular prosthesis formed of only air-dried EBM layer did not leak fluid or circulating blood cells. A vascular prosthesis formed of only freeze-dried EBM layer leaked fluid at a rate of about 6.6 μl/min/cm² and leaked cells. A freeze-dried harvested vessel leaked fluid at a rate of about 40.0 μl/min/cm² and leaked cells. An air-dried harvested vessel leaked fluid at a rate of about 75.0 μl/min/cm² and leaked cells.

The prosthesis can be used for a variety of applications, including, hemodialysis access, cardiac bypass, and peripheral arterial vessel bypass.

A number of embodiments of the invention have been described. Nevertheless, it will be understood that various modifications may be made without departing from the spirit and scope of the invention. Accordingly, other embodiments are within the scope of the following claims.

### EXAMPLE 2

A vessel prepared as described above can be cellularized using any of a variety of methods. For example, the freeze-dried vessel, optionally while still on the mandrel, can be immersed partially or completely in a solution containing culture medium and cells (and, optionally, collagen). The vessel is incubated in the cell-containing culturing medium under conditions suitable for culturing the cells (e.g., at 37° C. in a Co₂ atmosphere). The cells (e.g., smooth muscle cells) will penetrate the freeze-dried material forming the vessel. It can be desirable to allow the cells to attach and (optionally) proliferate until the layer of cells surrounding the vessel in 0.1 to 1.0 mm thick. The vessel can be placed under tension during or after this incubation in order to stretch the vessel somewhat.

### EXAMPLE 3

A vessel can produced by first forming a collagen foam tube (e.g., cylinder) and then allowing cells to grow into the collagen foam. Briefly, using an appropriate form a hollow tube of collagen foam is formed by filling the tube with hydrated collagen (among the useful types of collagen are those described in U.S. Pat. Nos. 5,562,946; 5,851,290; 5,911,942; and 6,705,850). The material is freeze-dried in the form to prepare a hollow tube of collagen foam. The hollow tube of collagen foam is transferred to a tubular mandrel (e.g., a Teflon® mandrel) that is then placed inside a hollow tube. A mixture of cells in culture medium (optionally including collagen) is introduced into the hollow tube so that cells can infiltrate the collagen foam. The collagen foam tube is incubated in contact with the cells and culture medium (e.g., at 37° C. in a Co₂ atmosphere). Over a period of days the ingrowth of cells causes the collagen foam tube to contract, particularly in a radial direction. This contraction causes the release of fluid from the foam resulting in a relatively thin-walled tube comprising collagen and cells. This process can take several days (e.g., about four days). The resulting thin-walled tube can be wrapped with collagen sheet (e.g., a sheet of meshed collagen). A preferred type of collagen is EBM, as described U.S. Pat. No. 6,696,074, hereby incorporated by reference). The meshed collagen can be meshed in a ratio of 1:1 to 1:6 (e.g., 1:3). The slits increasing in size with increase pf the second number in the ratio using a Brenner mesher, for example. The collagen can be provided with a line a hooks places at intervals along one edge. When the edges are brought together, the hooks can engage the slots created by the meshing. The collagen sleeve can extend several centimeters beyond each end of the vessel, so that it can serve as a holdfast, allowing the application of a steady but gentle stretch of the vessel. To better secure the meshed collagen, a thin cell-seeded gel (e.g., containing advential fibroblasts) may be cast around it, filling the open slits in the collagen sheet and binding to the subadjacent collagen gel.

What is claimed is:

1. A vascular prosthesis in the form of a tube comprising at least one layer of freeze-dried collagen-containing matrix surrounding at least one layer of air-dried collagen-containing matrix.

2. The vascular prosthesis of claim 1 comprising at least one layer of freeze-dried collagen-containing matrix surrounding at least two layers of air-dried collagen-containing matrix.

3. The vascular prosthesis of claim 1 comprising at least two layers of freeze-dried collagen-containing matrix surrounding at least two layers of air-dried collagen-containing matrix.

4. The vascular prosthesis of claim 2 wherein at least a portion of a first layer of air-dried collagen-containing matrix is mechanically joined by overlap to at least a portion of a second layer of air-dried collagen-containing matrix.

5. The vascular prosthesis of claim 3 wherein at least a portion of a first layer of freeze-dried collagen-containing matrix is mechanically joined to at least a portion of a second layer of freeze-dried collagen-containing matrix.

6. The vascular prosthesis of claim 4 wherein the first and second layers are mechanically joined by stitching.

7. The vascular prosthesis of claim 4 wherein the first and second layers are mechanically joined by stapling.

8. The vascular prosthesis of claim 1 having an inner diameter of no more than 4 mm.

9. The vascular prosthesis of claim 1 having an inner diameter of at least 4 mm.

10. The vascular prosthesis of claim 1 wherein the air-dried collagen-containing matrix comprises non-human, fetal or neo-natal tissue.

11. The vascular prosthesis of claim 1 wherein the freeze-dried collagen-containing matrix comprises non-human, fetal or neo-natal tissue.

12. The vascular prosthesis of claim 11 wherein the freeze-dried collagen-containing matrix comprises non-human, fetal or neo-natal tissue is bovine dermis that is substantially free of epidermal matter and subdermal matter.

13. A method for forming a vascular prosthesis comprising:
(a) wrapping a strip of rehydrated air-dried collagen-containing matrix around a mandrel to create at least one layer of rehydrated air-dried collagen-containing matrix;
(b) wrapping a strip of rehydrated freeze-dried collagen-containing matrix around the at one layer of rehydrated air-dried collagen-containing matrix to create at least one layer of rehydrated freeze-dried collagen-containing matrix surrounding the at least one layer of rehydrated air-dried collagen-containing matrix to create a vascular prosthesis; and
(c) removing the vascular prosthesis from the mandrel.

14. The method of claim 13 wherein step (a) comprises wrapping the strip of rehydrated air-dried collagen-containing matrix around a mandrel to create at least two layers of rehydrated air-dried collagen-containing matrix.

15. The method of claim 13 wherein step (b) comprises wrapping the strip of rehydrated freeze-dried collagen-containing matrix around the at least one layer of rehydrated air-dried collagen-containing matrix to create at least one layer of rehydrated freeze-dried collagen-containing matrix surrounding the at least one layer of rehydrated air-dried collagen-containing matrix.

16. The method of claim 13 wherein step (a) comprises wrapping a strip of rehydrated air-dried collagen-containing matrix around a mandrel to create at least two layers of rehydrated air-dried collagen-containing matrix.

17. The method of claim 14 wherein two or more of the at least two layers of rehydrated air-dried collagen-containing matrix are mechanically joined.

18. The method of claim 15 wherein two or more of the at least one layers of rehydrated freeze-dried collagen-containing matrix are mechanically joined.

19. The method of claim 17 wherein the layers are mechanically joined by stitching.

20. The method of claim 17 wherein the layers are mechanically joined by stapling.
